(19) United States
(12) Patent Application Publication
(10) Pub. No.: US 2026/0257982 A1
(43) Pub. Date: Sep. 3, 2026

(54) METHOD OF MANUFACTURING ACETIC ACID

(71) Applicants: Haruyuki NAKANISHI, Toyota-shi (JP); Yoshitsugu KOJIMA, Higashihiroshima-shi (JP)

(72) Inventors: Haruyuki NAKANISHI, Toyota-shi (JP); Yoshitsugu KOJIMA, Higashihiroshima-shi (JP)

(73) Assignees: TOYOTA JIDOSHA KABUSHIKI KAISHA, Aichi-ken (JP); HIROSHIMA UNIVERSITY, Hiroshima-Ken (JP)

(21) Appl. No.: 19/551,522

(22) Filed: Feb. 26, 2026

(30) Foreign Application Priority Data
Feb. 28, 2025 (JP) 2025-032364

(51) Int. Cl. C07C 51/15

(52) U.S. Cl. CPC C07C 51/15

(57) ABSTRACT

A method of manufacturing acetic acid by reducing carbon dioxide from the atmosphere includes: a step of carrying out a treatment of immersing a hydrogen storage alloy negative electrode, which is used in a nickel-hydrogen battery, in an alkaline electrolytic solution and thereafter drying the negative electrode; and a step of, by using an electrolysis cell having an alkaline electrolytic solution that can adsorb carbon dioxide, a positive electrode that is used in a nickel-hydrogen battery, the hydrogen storage alloy negative electrode after the treatment, and a power source connected to the positive electrode and the negative electrode, reducing the carbon dioxide by applying, to the positive electrode and the negative electrode, a potential difference such that a potential of the positive electrode becomes higher than a potential of the negative electrode, wherein the alkaline electrolytic solution of the electrolysis cell is a K₂CO₃ aqueous solution.

### CROSS-REFERENCE TO RELATED APPLICATION

This application is based on and claims priority under 35 USC 119 from Japanese Patent Application No. 2025-032364 filed on Feb. 28, 2025, the disclosure of which is incorporated by reference herein.

### BACKGROUND

### Technical Field

The present disclosure relates to a method of manufacturing acetic acid.

### Related Art

CO₂ (i.e., carbon dioxide) is a greenhouse gas that exists naturally in the atmosphere and is generated by burning fossil fuels. Due to increased human activity and energy demands, the amount of CO₂ in the atmosphere has increased, and it is thought that this is giving rise to global warming. Researchers worldwide are focusing on methods for removing CO₂ within the atmosphere. Various CO₂ converting methods including chemical methods, photocatalytic methods, and electrochemical methods are being researched widely. Among these methods, electrochemical methods are one type of method that can be carried out at room temperature.

There is known a method in which CO₂ is reduced by using an electrochemical cell, and an organic carboxylic acid (formic acid, acetic acid, oxalic acid or the like) is extracted in situ, wherein materials of the cathode that are preferable for electrochemically reducing CO₂ within an alkanolamine (specifically, monoethanolamine or the like) that absorbs CO₂ include copper, iron, silver, and oxides or alloys thereof (Japanese Patent Application National Publication No. 2021-516290).

There is also known a technique of, by using single crystals of copper in the cathode, generating acetic acid and formic acid by electrolysis of a KHCO₃ aqueous solution, wherein the proportion of acetic acid that is generated as compared with formic acid is 20%-30% (Selective Formation of C2 Compounds from Electrochemical Reduction of CO₂ at a Series of Copper Single Crystal Electrodes, J. Phys. Chem. B, 2002, 106(1), 15-17). There is known a technique of, by using boron-doped diamond, which has been modified by copper and gold particles, in the cathode and using platinum in the counter electrode, synthesizing formic acid and acetic acid by reducing a KCl aqueous solution in which CO₂ has been dissolved, wherein the proportion of the synthesized amount of acetic acid with respect to the synthesized amount of formic acid, i.e., the selection ratio, is approximately 3% (Electrochemical reduction of carbon dioxide to acetic acid on a Cu-Au modified boron-doped diamond electrode with a flow-cell system, RSC Advances, 2023, 13, 22061-22069).

### SUMMARY

In conventional methods, mainly copper is used as the cathode utilized in the electrochemical reaction, and platinum that is expensive is used as the anode, and the selection ratio of the generated acetic acid is 1 or less.

An object of an embodiment of the present disclosure is to provide a method of manufacturing carbon-neutral acetic acid efficiently and with a low environmental burden.

Means for addressing this topic include the following aspects.

A first aspect of the present disclosure provide a method of manufacturing acetic acid by reducing carbon dioxide from the atmosphere, the method including: carrying out a treatment of immersing a hydrogen storage alloy negative electrode, which is used in a nickel-hydrogen battery, in an alkaline electrolytic solution and then drying the negative electrode; and by using an electrolysis cell having an alkaline electrolytic solution that can adsorb carbon dioxide, a positive electrode that is used in a nickel-hydrogen battery, the hydrogen storage alloy negative electrode after the treatment, and a power source connected to the positive electrode and the negative electrode, reducing the carbon dioxide by applying, to the positive electrode and the negative electrode, a potential difference such that a potential of the positive electrode becomes higher than a potential of the negative electrode, wherein the alkaline electrolytic solution of the electrolysis cell is a K₂CO₃ aqueous solution.

A second aspect of the present disclosure provides the method of manufacturing acetic acid of the first aspect, wherein the hydrogen storage alloy negative electrode contains MmNi₅, and the positive electrode contains at least one of Ni(OH)₂ or NiOOH.

A third aspect of the present disclosure provides the method of manufacturing acetic acid of the first aspect or the second aspect, wherein the hydrogen storage alloy negative electrode after the treatment has a diffraction peak in a range of 2θ=32° to 33° in X-ray diffraction (XRD) measurement.

A fourth aspect of the present disclosure provides the method of manufacturing acetic acid of any one of the first aspect through the third aspect, wherein acetic acid and formic acid are manufactured, and a number of moles of the manufactured acetic acid is greater than a number of moles of the manufactured formic acid.

In accordance with an embodiment of the present disclosure, there is provided a method of manufacturing carbon-neutral acetic acid efficiently and with a low environmental burden.

### BRIEF DESCRIPTION OF THE DRAWINGS

FIG. 1 is a graph of X-ray diffraction results of respective MmNi₅-type hydrogen storage alloy cathodes of an Example and a Comparative Example.

### DETAILED DESCRIPTION

Embodiments of the present disclosure are described hereinafter with reference to the drawing.

In the present disclosure, numerical value ranges expressed by using “−” mean ranges in which the numerical values listed before and after the “−” are included as the minimum value and maximum value, respectively.

In numerical value ranges that are expressed in a stepwise manner in the present disclosure, the maximum value or the minimum value listed in a given numerical value range may be substituted by the maximum value or the minimum value of another numerical value range that is expressed in a stepwise manner. In the numerical value ranges put forth in the present disclosure, the maximum value or the minimum value listed in a given numerical value range may be substituted by a value set forth in the Examples.

In the present disclosure, “step” is not only an independent step and includes steps that, even in a case in which that step cannot be clearly distinguished from another step, achieve the intended object of that step.

In the present disclosure, combinations of two or more preferable aspects are more preferable aspects.

In the present disclosure, the “anode” in the electrolytic reaction can also be called the “positive electrode”, and the “cathode” can also be called the “negative electrode”.

### <Method of Manufacturing Acetic Acid>

A method of manufacturing acetic acid relating to the present disclosure is a method of manufacturing acetic acid by reducing carbon dioxide from the atmosphere, and includes a treating step and a reducing step. In the treating step, a treatment of immersing a hydrogen storage alloy negative electrode, which is used in a nickel-hydrogen battery, in an alkaline electrolytic solution and thereafter drying the negative electrode is carried out. In the reducing step, carbon dioxide is reduced by applying a potential difference, which is such that the potential of a positive electrode becomes higher than the potential of the negative electrode, to the positive electrode and the negative electrode by a power source by using an electrolysis cell. The electrolysis cell has the alkaline electrolytic solution, the positive electrode, the negative electrode and the power source. The alkaline electrolytic solution is a K₂CO₃ aqueous solution that can adsorb carbon dioxide. The positive electrode and the negative electrode respectively are electrodes that are used in a nickel-hydrogen battery. Before being used in the electrolysis, the negative electrode is subjected to a pre-treatment by the treating step. The power source is connected to the positive electrode and the negative electrode.

### (Treating Step)

In the treating step, it suffices for the hydrogen storage alloy negative electrode to be a negative electrode that contains a hydrogen storage alloy that is used in a nickel-hydrogen battery (i.e., a nickel-hydrogen secondary battery (Ni-MH)). From the standpoint of the environmental burden, the hydrogen storage alloy is preferably an AB₅ type hydrogen storage alloy that is widely used in nickel-hydrogen batteries, and more preferably is a hydrogen storage alloy whose raw material is a Misch metal, and more preferably contains MmNi₅. The hydrogen storage alloy negative electrode may be a hydrogen storage alloy negative electrode before being used in a battery, or may be a hydrogen storage alloy negative electrode contained in a nickel-hydrogen battery after usage thereof.

The alkaline electrolytic solution is preferably an alkaline electrolytic solution that is used in nickel-hydrogen batteries. The alkaline electrolytic solution may be an alkaline electrolytic solution before being used in a nickel-hydrogen battery, or may be an alkaline electrolytic solution after usage in a nickel-hydrogen battery. A single type of alkaline electrolytic solution may be used or plural types may be mixed together. The alkaline electrolytic solution may be the same type of electrolytic solution as alkaline electrolytic solutions that are used in electrolysis cells.

Examples of alkaline electrolytic solutions are potassium carbonate (K₂CO₃) aqueous solution, potassium hydroxide (KOH) aqueous solution, potassium bicarbonate (KHCO₃) aqueous solution, sodium hydroxide (NaOH) aqueous solution, sodium carbonate (Na₂CO₃) aqueous solution, and lithium hydroxide (LiOH) aqueous solution.

From the standpoint of the selection ratio of the acetic acid, the alkaline electrolytic solution preferably includes KOH, KHCO₃ and K₂CO₃. Note that, by adsorbing CO₂ that is in the atmosphere, a KOH aqueous solution becomes a K₂CO₃ aqueous solution or a potassium bicarbonate (KHCO₃) aqueous solution, and therefore, an aqueous solution obtained by causing a KOH aqueous solution to contact the atmosphere may be used as the alkaline electrolytic solution.

The method of immersing the hydrogen storage alloy negative electrode in an alkaline electrolytic solution is not particularly limited. It suffices for some of or all of the hydrogen storage alloy negative electrode to be immersed in the alkaline electrolytic solution. If the hydrogen storage alloy negative electrode is a hydrogen storage alloy negative electrode that is contained in a nickel-hydrogen battery after usage thereof, the hydrogen storage alloy negative electrode has already been immersed in an alkaline electrolytic solution. Therefore, a hydrogen storage alloy negative electrode that has been removed from a nickel-hydrogen battery after usage thereof may be used as is.

The hydrogen storage alloy negative electrode that has been immersed in the alkaline electrolytic solution is dried thereafter. The drying does not have to be complete drying of an extent such that all of the moisture is removed, and may be drying of an extent such that the alkaline electrolytic solution that has adhered to the hydrogen storage alloy negative electrode is concentrated.

The present inventors discovered that, by using, in an electrolysis cell, a dried hydrogen storage alloy negative electrode to which an alkaline electrolytic solution has adhered, the resistance of the hydrogen storage alloy negative electrode in an electrolytic solution is greatly lowered. Although the reason for this is not clear, it is assumed that the surface of the hydrogen storage alloy negative electrode is activated by changes in the surface state, and acetic acid is selectively manufactured in electrolysis. One such change in the surface state is assumed to be that at least some of the components of the alkaline electrolytic solution become crystal and exist on the surface. It is assumed that, due thereto, acetic acid ions are selectively formed in the CO₂ reduction reaction due to an environment such as a locally strong alkaline environment being maintained.

From the standpoint of the selection ratio of the acetic acid, the drying temperature is preferably a relatively low temperature. As an example, drying is carried out at 80° C. for several hours in the atmosphere. It suffices for the drying to be such that the hydrogen storage alloy negative electrode to which the alkaline electrolytic solution has adhered is dried, and the conditions thereof are not limited, but it is surmised that, by drying at such a relatively low temperature, crystals that adhere relatively uniformly will form.

Due to the treating step, the hydrogen storage alloy negative electrode after the treatment preferably has a diffraction peak in the range of 2θ=32°−33° in X-ray diffraction (XRD) measurement. A diffraction peak in the range of 2θ=32°−33° is a diffraction peak that does not exist in the X-ray diffraction (XRD) measurement results of the hydrogen storage alloy negative electrode before the treatment, and is newly contained in the hydrogen storage alloy negative electrode after the treatment. It is assumed that, due to the treating step such as that described above, crystals of KOH that is a component of the alkaline electrolytic solution exist at a portion of the surface of the hydrogen storage alloy negative electrode after the treatment, and a diffraction peak in the range of 2θ=32°−33° newly appears as a diffraction peak deriving from the crystals of KOH. Note that the hydrogen storage alloy negative electrode after the treatment also has a peak deriving from the pre-treatment hydrogen storage alloy itself.

### (Reducing Step)

An electrolysis cell is used in the reducing treatment. The alkaline electrolytic solution that the electrolysis cell has is an aqueous solution that can adsorb carbon dioxide (CO₂), and, from the standpoint of the selection ratio of the acetic acid, is a K₂CO₃ aqueous solution. The alkaline electrolytic solution that the electrolysis cell has may be an Na₂CO₃ aqueous solution, or may be a mixture of a K₂CO₃ aqueous solution and an Na₂CO₃ aqueous solution, or may be a KOH aqueous solution. The alkaline electrolytic solution that the electrolysis cell has may contain components other than K₂CO₃.

In the presence of water, potassium carbonate (K₂CO₃) reacts with CO₂ (400 ppm, 40 Pa) that is within the atmosphere, and generates potassium bicarbonate (KHCO₃). The reaction formula is shown by formula (2).

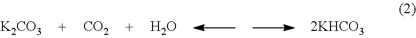

In the Van't Hoff equation expressed by following formula (3), ΔH⁰ is the standard enthalpy change (−96.1 kJ/mol CO₂), and ΔS⁰ is the standard entropy change (−208 J/Kmol CO₂). P⁰ is replaced with atmospheric pressure (0.1 MPa), R is replaced with the gas constant 8.314 JK⁻¹ mol⁻¹, and T is replaced with temperature (298K). As a result, pressure P is 0.112 Pa, and the equilibrium concentration of CO₂ absorption is 1.12 ppm. Therefore, carbon dioxide (400 ppm) that is within the atmosphere can be absorbed, and potassium bicarbonate (KHCO₃) is electrochemically used as a CO₂ source. Thereafter, the potassium bicarbonate (KHCO₃) is regenerated into potassium carbonate (K₂CO₃).

$$\begin{matrix}[ Formula 1 ] & \\ ln ( \frac{P}{{P}_{0}} ) = \frac{Δ {H}^{0}}{R T} - \frac{Δ {S}^{0}}{R} & ( 3 )\end{matrix}$$

Due to a KOH aqueous solution adsorbing CO₂ that is within the atmosphere, it becomes a K₂CO₃ aqueous solution or a potassium bicarbonate (KHCO₃) aqueous solution. Therefore, a KOH aqueous solution may be used by being made to contact the atmosphere. KOH absorbs CO₂ (400 ppm) that is within the atmosphere, and potassium carbonate (K₂CO₃) and water are partially generated. The reaction formula is expressed by formula (4).

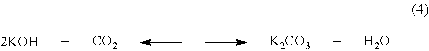

In the Van′t Hoff equation expressed by above formula (3), ΔH⁰ is the standard enthalpy change (−194 KJ/mol CO₂), and ΔS° is the standard entropy change (−151 J/Kmol CO₂). P° is replaced with atmospheric pressure (0.1 MPa), R is replaced with the gas constant 8.314 JK⁻¹ mol⁻¹, and T is replaced with temperature (298K). As a result, the pressure P is 0.112 Pa, the equilibrium concentration of CO₂ absorption is 7.92×10⁻²¹ ppm, and KOH can absorb carbon dioxide that is within the atmosphere. Because this K₂CO₃ is thermodynamically stable, it can be thought that, from the standpoint of the selection ratio of the acetic acid, the above-described K₂CO₃ aqueous solution utilizes K₂CO₃ as the CO₂ source more effectively than a KOH aqueous solution, and is preferable as the alkaline electrolytic solution. Note that, in a case of using a KOH aqueous solution as the alkaline electrolytic solution in the electrolysis cell, electrolysis may be carried out by making the potential difference great or the like.

The alkaline electrolytic solution may be an electrolytic solution contained in a nickel-hydrogen battery (i.e., a nickel-hydrogen secondary battery (Ni-MH)). The electrolytic solution may be an electrolytic solution that is contained in a nickel-hydrogen battery before use, or may be an electrolytic solution that is contained in a nickel-hydrogen battery after use. Minute amounts of elements or compounds other than those described above may be contained in the alkaline electrolytic solution.

As the electrodes that the electrolysis cell has, a positive electrode that is used in nickel-hydrogen batteries is used as the positive electrode. The positive electrode preferably contains at least one of Ni(OH)₂ or NiOOH. An element other than Ni(OH)₂ and NiOOH, such as Co or Fe, may be added to the positive electrode. The positive electrode may be a positive electrode that is contained in a nickel-hydrogen battery before use, or may be a positive electrode that is contained in a nickel-hydrogen battery after use.

From the standpoint of the selection ratio of the acetic acid, it is preferable to carry out a treating step, which is similar to that of the negative electrode, on the positive electrode. The treatment carried out on the positive electrode is similar to the treatment carried out on the negative electrode.

It suffices for the power source to be able to carry out electrolysis using an electrolysis cell, and the power source is not limited. Carbon dioxide is reduced due to a potential difference, which is such that the potential of the positive electrode becomes higher than the potential of the negative electrode, being applied by the power source to the positive electrode and the negative electrode. A power source that does not use fossil fuels or the like is preferably used as the power source. This is because acetic acid, which is a raw material of fuel, can be manufactured from carbon dioxide within the atmosphere with a smaller environment burden.

The electrolysis cell carries out an electrolysis in a state in which the alkaline electrolytic solution and the atmosphere can contact one another. Due thereto, the CO₂ that has been adsorbed by the alkaline electrolytic solution can be reduced electrochemically. It is thought that, in the method of manufacturing acetic acid of the present disclosure, acetic acid ions are generated by the reduction of CO₂, and most of the acetic acid exists in the alkaline electrolytic solution as potassium acetate. Acetic acid can be recovered by recovering the alkaline electrolytic solution that contains potassium acetate, and neutralizing the electrolytic solution.

In the method of manufacturing acetic acid of the present disclosure, other ions such as formic acid ions may be generated in addition to acetic acid ions. In the method of manufacturing acetic acid of the present disclosure, it is preferable that acetic acid and formic acid are manufactured, and that the number of moles of the manufactured acetic acid is greater than the number of moles of the manufactured formic acid. It is more preferable that the number of moles of the manufactured acetic acid is 5 times or more greater than the number of moles of the manufactured formic acid. Note that oxygen is generated from the positive electrode by electrolysis. The generated oxygen may be released into the atmosphere, or may be recovered and used.

Selection ratio S, which is the amount of acetic acid that is generated with respect to the amount of formic acid that is generated, can be calculated by following formula (1) from generated amount Ma of acetic acid and generated amount Mf of formic acid that are measured. Note that the generated amounts may be measured as masses.

$$S = M a / M f \qquad ( 1 )$$

According to the method for producing acetic acid of the present disclosure, acetic acid, which serves as a raw material for, for example, vinegar, polymers, and ethanol as a fuel, can be selectively produced by electrolysis from carbon dioxide present in the atmosphere. The electrolytic cell used for the electrolysis may use electrodes and an electrolytic solution taken from a used nickel-metal hydride battery. The method for producing acetic acid of the present disclosure can be implemented by, for example, opening a used nickel-metal hydride battery, removing the negative electrode therefrom, subjecting the negative electrode to the treating step and thereafter returning the negative electrode to the battery, and applying a potential difference between the two electrodes. Further, even in cases of using an electrolytic solution or a positive electrode or a negative electrode that has not been used in a nickel-metal hydride battery, existing components can be used as is. Accordingly, by the method for producing acetic acid of the present disclosure, carbon-neutral acetic acid can be produced efficiently and with a low environmental burden.

### EXAMPLES

The present disclosure is described in further detail hereinafter by way of Examples. The Examples of the present specification are exemplary and are not intended to limit the present disclosure. Persons having ordinary skill in the field relating to the present disclosure can appropriately revise, modify and substitute materials, compositions, manufacturing methods and objects of application, without departing from the spirit and the scope of the present invention. Note that “Ni(OH)₂/NiOOH positive electrode” means that the positive electrode contains at least one of Ni(OH)₂ and NiOOH, and may contain both.

### (Measuring Method)

In the following Examples and Comparative Examples, after electrolysis is carried out under predetermined conditions by using an electrolysis cell, ion chromatography is carried out, and the generated amount Ma of acetic acid and the generated amount Mf of formic acid are measured. The selection ratio S, which is the generated amount of acetic acid with respect to the generated amount of formic acid, is calculated from these manufactured amounts by using above formula (1). Note that the manufactured amounts are measured as masses. The results of measurement and results of calculation are listed in Table 1.

### Example 1

A nickel-hydrogen battery was disassembled, and an Ni(OH)₂/NiOOH positive electrode and an MmNi₅ type hydrogen storage alloy negative electrode were removed. In the treating step, the positive electrode and negative electrode were subjected to a heat treatment in air for 3 hours at 80° C. Using the heat-treated positive electrode as the anode and the heat-treated negative electrode as the cathode, an electrolysis test in a K₂CO₃ aqueous solution was carried out in atmosphere at 2 V for 400 hours. The current was 15 mA-6 mA. Thereafter, ion chromatography was carried out by using the electrolytic solution as the subject thereof.

### Example 2

A nickel-hydrogen battery was disassembled, and an Ni(OH)₂/NiOOH positive electrode and an MmNi₅ type hydrogen storage alloy negative electrode were removed. In the treating step, the positive electrode and negative electrode were subjected to a heat treatment in air for 3 hours at 80° C. Using the heat-treated positive electrode as the anode and the heat-treated negative electrode as the cathode, an electrolysis test in a K₂CO₃ aqueous solution was carried out in atmosphere at 2 V for 250 hours. The current was 15 mA-7 mA. Thereafter, ion chromatography was carried out by using the electrolytic solution as the subject thereof.

### Example 3

A nickel-hydrogen battery was disassembled, and an Ni(OH)₂/NiOOH positive electrode and an MmNi₅ type hydrogen storage alloy negative electrode were removed. In the treating step, the positive electrode and negative electrode were subjected to a heat treatment in air for 3 hours at 80° C. Using the heat-treated positive electrode as the anode and the heat-treated negative electrode as the cathode, an electrolysis test in a K₂CO₃ aqueous solution was carried out in atmosphere at 2 V for 100 hours. The current was 15 mA-8 mA. Thereafter, ion chromatography was carried out by using the electrolytic solution as the subject thereof.

### Example 4

A nickel-hydrogen battery was disassembled, and an Ni(OH)₂/NiOOH positive electrode and an MmNi₅ type hydrogen storage alloy negative electrode were removed. The negative electrode was subjected to a heat treatment in air for 3 hours at 80° C. Using the positive electrode as the anode and the heat-treated negative electrode as the cathode, an electrolysis test in a K₂CO₃ aqueous solution was carried out in atmosphere at 2 V for 100 hours. The current was 9 mA-4 mA. Thereafter, ion chromatography was carried out by using the electrolytic solution as the subject thereof.

### Comparative Example 1

A nickel-hydrogen battery was disassembled, and an Ni(OH)₂/NiOOH positive electrode and an MmNi₅ type hydrogen storage alloy negative electrode were removed. The positive electrode was subjected to a heat treatment in air for 3 hours at 80° C. Using the heat-treated positive electrode as the anode and the negative electrode as the cathode, an electrolysis test in a K₂CO₃ aqueous solution was carried out in atmosphere at 2 V for 100 hours. The current was 7 mA-3 mA. Thereafter, ion chromatography was carried out by using the electrolytic solution as the subject thereof.

### Comparative Example 2

A nickel-hydrogen battery was disassembled, and an Ni(OH)₂/NiOOH positive electrode and an MmNi₅ type hydrogen storage alloy negative electrode were removed. Using the Ni(OH)₂/NiOOH positive electrode as the anode and the MmNi₅ type hydrogen storage alloy negative electrode as the cathode, an electrolysis test in a K₂CO₃ aqueous solution was carried out in atmosphere at 2 V for 100 hours. The current was 5 mA-2 mA. Thereafter, ion chromatography was carried out by using the electrolytic solution as the subject thereof.

### Comparative Example 3

A nickel-hydrogen battery was disassembled, and an Ni(OH)₂/NiOOH positive electrode and an MmNi₅ type hydrogen storage alloy negative electrode were removed. The positive electrode and negative electrode were subjected to a heat treatment in air for 3 hours at 80° C. Using the heat-treated positive electrode as the anode and the heat-treated negative electrode as the cathode, an electrolysis test in a K₂CO₃ aqueous solution was carried out in nitrogen at 2 V for 100 hours. The current was 15 mA-8 mA. Thereafter, ion chromatography was carried out by using the electrolytic solution as the subject thereof.

### Comparative Example 4

A nickel-hydrogen battery was disassembled, and an Ni(OH)₂/NiOOH positive electrode and an MmNi₅ type hydrogen storage alloy negative electrode were removed. The positive electrode and negative electrode were subjected to a heat treatment in air for 3 hours at 80° C. Using the heat-treated positive electrode as the anode and the heat-treated negative electrode as the cathode, an electrolysis test in a KOH aqueous solution (6 mol/L) was carried out in atmosphere at 2 V for 100 hours. The current was 15 mA-8 mA. Thereafter, ion chromatography was carried out by using the electrolytic solution as the subject thereof.

### Comparative Example 5

A nickel-hydrogen battery was disassembled, and an Ni(OH)₂/NiOOH positive electrode and an MmNi₅ type hydrogen storage alloy negative electrode were removed. The positive electrode and negative electrode were subjected to a heat treatment in air for 3 hours at 80° C. Using the heat-treated positive electrode as the anode and the heat-treated negative electrode as the cathode, an electrolysis test in a KOH aqueous solution (6 mol/L) was carried out in nitrogen at 2 V for 100 hours. The current was 15 mA-8 mA. Thereafter, ion chromatography was carried out by using the electrolytic solution as the subject thereof.

TABLE 1

| | | | | acetic | formic | | | | |
|---|---|---|---|---|---|---|---|---|---|
| | | | | acid | acid | selec- | | | |
| | | electro- | | amount | amount | tion | voltage | current | time |
| | electrodes | lyte | atmosphere | (mg/L) | (mg/L) | ratio | (V) | (mA) | (h) |
| Ex. 1 | heat-treated | K₂CO₃ | in atmosphere | 120 | 3 | 40 | 2 | 15-6 | 400 |
| | anode, | | | | | | | | |
| | heat-treated | | | | | | | | |
| | cathode | | | | | | | | |
| Ex. 2 | heat-treated | K₂CO₃ | in atmosphere | 15 | 3 | 5 | 2 | 15-7 | 250 |
| | anode, | | | | | | | | |
| | heat-treated | | | | | | | | |
| | cathode | | | | | | | | |
| Ex. 3 | heat-treated | K₂CO₃ | in atmosphere | 11 | 1 or less | 10 or | 2 | 15-8 | 100 |
| | anode, | | | | | more | | | |
| | heat-treated | | | | | | | | |
| | cathode | | | | | | | | |
| Ex. 4 | untreated anode, | K₂CO₃ | in atmosphere | 5 | 1 or less | 5 or | 2 | 9-4 | 100 |
| | heat-treated | | | | | more | | | |
| | cathode | | | | | | | | |
| Comp. | heat-treated | K₂CO₃ | in atmosphere | 1 | 1 | 1 | 2 | 7-3 | 100 |
| Ex. 1 | anode, | | | | | | | | |
| | untreated | | | | | | | | |
| | cathode | | | | | | | | |
| Comp. | untreated anode, | K₂CO₃ | in atmosphere | 1 | 1 | 1 | 2 | 5-2 | 100 |
| Ex. 2 | untreated | | | | | | | | |
| | cathode | | | | | | | | |
| Comp. | heat-treated | K₂CO₃ | in nitrogen | 2 | 1 | 2 | 2 | 15-8 | 100 |
| Ex. 3 | anode, | | | | | | | | |
| | heat-treated | | | | | | | | |
| | cathode | | | | | | | | |
| Comp. | heat-treated | KOH | in atmosphere | 2 | 1 | 2 | 2 | 15-8 | 100 |
| Ex. 4 | anode, | | | | | | | | |
| | heat-treated | | | | | | | | |
| | cathode | | | | | | | | |
| Comp. | heat-treated | KOH | in nitrogen | 1 or less | 1 or less | — | 2 | 15-8 | 100 |
| Ex. 5 | anode, | | | | | | | | |
| | heat-treated | | | | | | | | |
| | cathode | | | | | | | | |

As shown in Table 1, in Examples 1-3, the amount of acetic acid generated by electrolyzing a K₂CO₃ aqueous solution in atmosphere by using heat-treated Ni(OH)₂/NiOOH as the anode and a heat-treated MmNi₅ type hydrogen storage alloy as the cathode was 5 mg/L-120 mg/L, and the selection ratio (acetic acid amount/formic acid amount) was 5-40. In Example 4, even by using untreated Ni(OH)₂/NiOOH as the anode and a heat-treated MmNi₅ type hydrogen storage alloy cathode, acetic acid was generated by electrolyzing a K₂CO₃ aqueous solution in atmosphere, and the selection ratio (acetic acid amount/formic acid amount) was 5 or more. On the other hand, as shown by Comparative Example 1 and Comparative Example 2, hardly any acetic acid was generated in cases in which the MmNi₅ type hydrogen storage alloy was not heat-treated.

In Comparative Example 3, when a K₂CO₃ aqueous solution was electrolyzed in a nitrogen atmosphere by using a heat-treated Ni(OH)₂/NiOOH as the anode and a heat-treated MmNi₅ type hydrogen storage alloy as the cathode, the generated amount of acetic acid was reduced greatly. On the other hand, in the atmosphere such as in Example 3, the generated amount of acetic acid increased. From these, it was confirmed that the potassium carbonate was converted into acetic acid after absorbing carbon dioxide that was within the atmosphere, as shown by above formula (2).

As demonstrated in Comparative Example 4, the generated amount of acetic acid was reduced greatly when a KOH aqueous solution was used as the electrolytic solution. In Comparative Example 5, when a KOH aqueous solution was electrolyzed in a nitrogen atmosphere by using a heat-treated Ni(OH)₂/NiOOH as the anode and a heat-treated MmNi₅ type hydrogen storage alloy as the cathode, acetic acid was not generated because CO₂ did not exist.

### Example 5

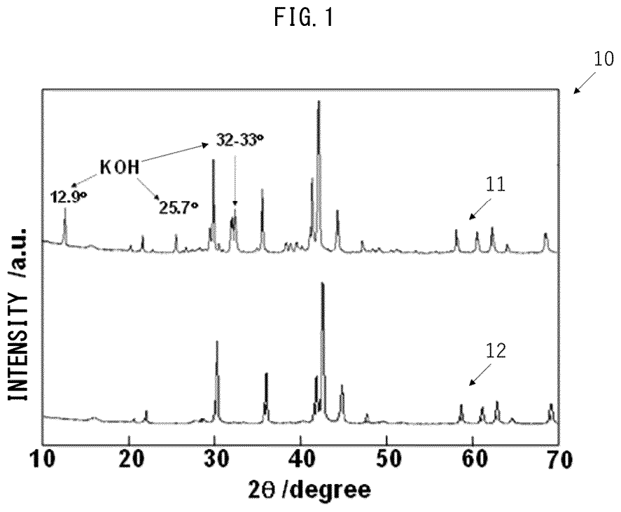

An MmNi₅ type hydrogen storage alloy cathode that had been removed from a nickel-hydrogen battery was subjected to a heat treatment in atmosphere for 3 hours at 80° C. The X-ray diffraction intensity curve of the heat-treated MmNi₅ type hydrogen storage alloy cathode is shown in FIG. 1.

### Comparative Example 6

The X-ray diffraction intensity curve of an MmNi₅ type hydrogen storage alloy cathode, which had been removed from a nickel-hydrogen battery and thereafter was washed but on which no heat treatment was carried out, is shown in FIG. 1.

Due to the MmNi₅ type alloy being heat-treated in Example 5, a new peak, which did not exist in Comparative Example 6, arose at 20=32° to 33°. It is thought that this peak is the crystallized KOH of the electrolyte that was used in the nickel-hydrogen battery. Note that new peaks existed at 12.9° and 25.7° as well, and it is thought that these also are crystallized KOH of electrolytes used in the nickel-hydrogen battery.

What is claimed is:

1. A method of manufacturing acetic acid by reducing carbon dioxide from the atmosphere, the method comprising:
carrying out a treatment of immersing a hydrogen storage alloy negative electrode, which is used in a nickel-hydrogen battery, in an alkaline electrolytic solution and then drying the negative electrode; and
by using an electrolysis cell having an alkaline electrolytic solution that can adsorb carbon dioxide, a positive electrode that is used in a nickel-hydrogen battery, the hydrogen storage alloy negative electrode after the treatment, and a power source connected to the positive electrode and the negative electrode, reducing the carbon dioxide by applying, to the positive electrode and the negative electrode, a potential difference such that a potential of the positive electrode becomes higher than a potential of the negative electrode,
wherein the alkaline electrolytic solution of the electrolysis cell is a K₂CO₃ aqueous solution.

2. The method of manufacturing acetic acid of claim 1, wherein:
the hydrogen storage alloy negative electrode contains MmNi₅, and
the positive electrode contains at least one of Ni(OH)₂ or NiOOH.

3. The method of manufacturing acetic acid of claim 1, wherein the hydrogen storage alloy negative electrode after the treatment has a diffraction peak in a range of 2θ=32° to 33° in X-ray diffraction (XRD) measurement.

4. The method of manufacturing acetic acid of claim 1, wherein:
acetic acid and formic acid are manufactured, and
a number of moles of the manufactured acetic acid is greater than a number of moles of the manufactured formic acid.
